(19) United States
(12) Patent Application Publication
(10) Pub. No.: US 2019/0175601 A1
(43) Pub. Date: Jun. 13, 2019

(54) COMPOSITION FOR INHIBITING OR ALLEVIATING ITCHING INCLUDING RIBOFLAVIN

(71) Applicant: INDUSTRY-UNIVERSITY COOPERATION FOUNDATION HANYANG UNIVERSITY, Seoul (KR)

(72) Inventors: Sung Jun JUNG, Gyeonggi-do (KR); Yoon Hee KANG, Gyeonggi-do (KR); Pyung-sun CHO, Seoul (KR); Han Kyu LEE, Gyeonggi-do (KR)

(21) Appl. No.: 16/278,557

(22) Filed: Feb. 18, 2019

Related U.S. Application Data
(63) Continuation-in-part of application No. PCT/KR2017/009022, filed on Aug. 18, 2017.

(30) Foreign Application Priority Data
Aug. 18, 2016 (KR) 10-2016-0104846

(51) Int. Cl. A61K 31/525; A61K 8/49; A61K 9/00; A61Q 19/00; A61P 17/04

(52) U.S. Cl. CPC A61K 31/525; A61K 8/4953; A61P 17/04; A61K 9/0053; A61Q 19/00; A61K 9/0021

(57) ABSTRACT

Provided is a composition for inhibiting or alleviating itching, which includes riboflavin or riboflavin analog.

The riboflavin or riboflavin analog are very effective in inhibiting or alleviating itching, scalp itching, or allergic itching by inhibiting itching information transmission through blocking of a current generated by histamine, which is a major factor of itching.

### CROSS-REFERENCE TO RELATED APPLICATION

This application claims priority to and is a Continuation-In-Part of PCT/KR2017/009022 (WO2018/034539), filed Aug. 18, 2017, entitled “COMPOSITION COMPRISING RIBOFLAVIN FOR SUPPRESSING OR ALLEVIATING ITCHING”, which application claims priority to and the benefit of Korean Patent Application No. 10-2016-0104846, filed on Aug. 18, 2016, the disclosures of which are incorporated herein by reference in their entirety.

### BACKGROUND

### 1. Field of the Invention

The present invention relates to a composition for inhibiting or alleviating itching, which includes riboflavin or a riboflavin analog.

### 2. Discussion of Related Art

In recent years, due to social and environmental changes such as urbanization, modernization, environmental pollution, and the like along with rapid growth, the number of patients with unidentified pruritus tends to continuously increase. Itching or pruritus is defined as an unpleasant sensation that causes an urge to scratch or rub the skin. The itching or pruritus may also be caused by stimulation such as light contact, temperature changes, stress, and the like, chemical, physical, and electrical stimulation, and the like, and it may be accompanied by an itching reflex (Non-Patent Document 1). In addition, itching or pruritus is known as a major symptom of patients with a skin disease such as atopy, psoriasis, or contact dermatitis, or possessors of sensitive skin. It is known that itching is caused by activation of specific immune cells (T cells, macrophages, and the like) in the body and induction of the secretion of histamine, which is a mediator of itching, by various cytokines (TNF-α, IL-2, and the like) (Non-Patent Documents 2 and 3). Itching is often an important symptom of a systemic disease, and in most cases, it is difficult to control itching since it is due to multifactorial symptoms including both an itching sensory factor such as skin dryness and a neurological factor. In addition, itching is the most common symptom in dermatology and is a factor that severely lowers the quality of patient life (Non-Patent Documents 4 and 5). However, it is difficult to identify the precise mechanism of a cause of pruritus, so appropriate treatment for pruritus is lacking.

Recently, thanks to the development of neurophysiological research techniques and molecular biology, many facts about the occurrence of itching and delivery mechanisms thereof have been revealed. Generally, itching is transmitted to the anhydrous afferent nerve via C-fiber nociceptors, and pruritogens secreted at C-fiber ends, such as substance P, calcitonin-gene related peptides (CGRPs), and the like are known to trigger itching by binding to respective receptors (Non-Patent Documents 6 and 7). These pruritogens have been reported to induce the release of histamine, which is another mediator of itching, by activating mast cells (Non-Patent Document 8). These study results suggest new therapies in the future, but effective therapeutic agents are still lacking and only various topical anti-inflammatory agents have been proposed. In addition, accurate diagnosis for itching and the development of therapeutic agents therefor have not yet been achieved due to the complicated and multistage mechanism of pruritus.

As currently used itching therapies, topical and systemic agents, such as steroids, antihistamines, immunosuppressive agents, and the like, are used. Topical antipruritic agents consist mostly of a combination of salicylic acid (anti-inflammatory agent), camphor, menthol, an H1 antihistamine agent (dyphenhydramine), a local anesthetic (cinchocaine), an adrenocortical hormone analogue (glycyrrhizic acid), enoxolone (18-beta glycyrrhetinic acid), calamine ((ZnOH)₂SiO₃, non-specific skin sedative), crotamiton (scabicide), phenol, and the like. However, the aforementioned topical antipruritic agents are very insufficient in effectively inhibiting intractable itching caused by complicated combinations of various factors such as allergic responses, e.g., itching of atopic dermatitis, nerve stimulation responses, immune responses, and the like. Therefore, to date, adrenocortical steroids, which relieve accompanying secondary itching by non-specifically and strongly suppressing inflammatory responses and immune responses of the skin, are widely used for antipruritic effects. However, adrenocortical steroids are not fundamentally direct antipruritic agents, and have been found to be effective and safe for short-term treatment for 2 to 4 weeks when used, but stability and effects thereof cannot be guaranteed in long-term treatment for more than 1 year. In long-term use, severe side effects such as resistance expression, skin atrophy, striae distensae, metabolic disorders, skin discoloration, skin thinning, stretch marks, and the like occur, and thus adrenocortical steroids are not suitable for use in inhibiting itching. In addition, itching-inhibiting or alleviating ingredients cause problems such as drowsiness or reduced immunity when orally administered, have a skin safety problem when locally administered, and mostly have problems in terms of preparation stability when mixed with a skin composition, and thus the use thereof is limited (Non-Patent Document 9).

The goal of treatment for itching is to stop the symptoms, and therefore, it is important to develop an antipruritic agent capable of effectively controlling intractable itching. There is also growing interest in natural extracts including various active ingredients and having various functions.
(Non-Patent Document 1) Ikoma A, Steinhoff M, Stander S, Yosipovitch G, Schmelz M. The neurobiology of itch. Nat Rev Neurosci. 2006 July; 7(7):535-47.
(Non-Patent Document 2) Radossi P, Tison T, Vianello F, Dazzi F. Br J Haematol. Intractable pruritus in non-Hodgkin lymphoma/CLL: rapid response to IFN alpha. Br J Haematol. 1996 September; 94(3):579.
(Non-Patent Document 3) Nielsen H J, Petersen L J, Skov P S. Human, recombinant interleukin-2 induces in vitro histamine release in a dose-dependent manner. Cancer Biother. 1995 Winter; 10(4):279-86.
(Non-Patent Document 4) Sheehan-Dare R A, Henderson M J, Cotterill J A. Anxiety and depression in patients with chronic urticaria and generalized pruritus. Br J Dermatol. 1990 December; 123(6):769-74.
(Non-Patent Document 5) Hashiro M, Okumura M. Anxiety, depression and psychosomatic symptoms in patients with atopic dermatitis: comparison with normal controls and among groups of different degrees of severity. J Dermatol Sci. 1997 January; 14(1):63-7.
(Non-Patent Document 6) Kuraishi Y, Nagasawa T, Hayashi K, Satoh M. Scratching behavior induced by pruritogenic but not algesiogenic agents in mice. Eur J Pharmacol. 1995 Mar. 14; 275(3):229-33.
(Non-Patent Document 7) Andoh T, Nagasawa T, Satoh M, Kuraishi Y. Substance P induction of itch-associated response mediated by cutaneous NK1 tachykinin receptors in mice. J Pharmacol Exp Ther. 1998 September; 286(3):1140-5.
(Non-Patent Document 8) Toyoda M, Makino T, Kagoura M, Morohashi M. Immunolocalization of substance P in human skin mast cells. Arch Dermatol Res. 2000 August; 292(8):418-21.
(Non-Patent Document 8) Chang-jong Kim, Pathopsychology, Hanlim Industrial Co., 1988, p 61-69

### SUMMARY OF THE INVENTION

The present invention provides a composition that is effective in inhibiting or alleviating itching.

According to an aspect of the present invention, there is provided a composition for inhibiting or alleviating itching, which includes riboflavin represented by Formula 1 below or a riboflavin analog represented by Formula 2 below:

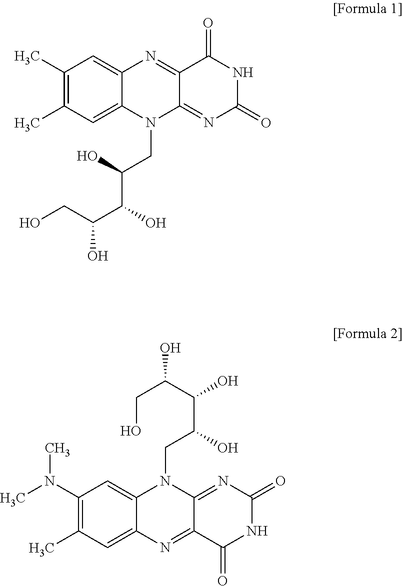 [SMILES: Cc1cc2nc3c(=O)[nH]c(=O)nc-3n(C[C@H](O)[C@H](O)[C@H](O)CO)c2cc1C ; Cc1cc2nc3c(=O)[nH]c(=O)nc-3n(C[C@@H](O)[C@@H](O)[C@@H](O)CO)c2cc1N(C)C]

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a graph showing evaluation results of histamine-induced itching by riboflavin according to concentration;

FIG. 2 is a graph showing evaluation results of histamine-induced itching upon co-administration of riboflavin and histamine;

FIG. 3 is a view illustrating the blockage of nerve activity when histamine was administered to the skin;

FIG. 4 is a graph showing results of confirming whether a current induced by histamine was inhibited by riboflavin using a membrane voltage clamp method;

FIGS. 5 to 7 are graphs showing a capsaicin current blocking effect of riboflavin; and

FIG. 8 is a graph showing evaluation results of histamine-induced itching by roseoflavin.

### DETAILED DESCRIPTION OF EXEMPLARY EMBODIMENTS

Exemplary embodiments of the present invention will be described in detail below with reference to the accompanying drawings. While the present invention is shown and described in connection with exemplary embodiments thereof, it will be apparent to those skilled in the art that various modifications can be made without departing from the spirit and scope of the invention.

Hereinafter, configurations of the present invention will be described in detail.

As can be confirmed in the following examples, as a result of performing an itch-inducing animal behavioral model test, a calcium response experiment, and an electrophysiological experiment, riboflavin or an analog thereof exhibited a potent effect of inhibiting and alleviating itching.

Therefore, the present invention provides a composition for inhibiting or alleviating itching, which includes riboflavin represented by Formula 1 below or an analog thereof represented by Formula 2 below:

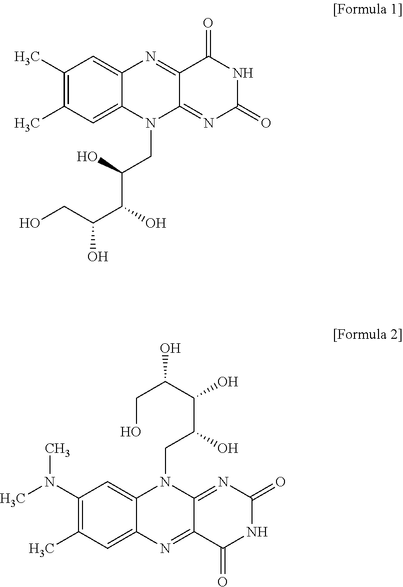 [SMILES: Cc1cc2nc3c(=O)[nH]c(=O)nc-3n(C[C@H](O)[C@H](O)[C@H](O)CO)c2cc1C ; Cc1cc2nc3c(=O)[nH]c(=O)nc-3n(C[C@@H](O)[C@@H](O)[C@@H](O)CO)c2cc1N(C)C]

The riboflavin is vitamin B₂ (C₁₇H₂₀N₄O₆, MW 376.36).

In addition, the riboflavin analog of the present invention, i.e., a compound represented by Formula 2 below may be roseoflavin (8-demethyl-8-(dimethylamino)-riboflavin, 8-dimethylaminoriboflavin). The roseoflavin has a MW of 405.41 and is a lipid-soluble form of riboflavin.

As the riboflavin or an analog thereof, a synthesized or commercially available compound may be used.

The composition of the present invention may include a pharmaceutically acceptable salt of the compound of Formula 1 or 2.

The pharmaceutically acceptable salt may be an acid addition salt formed using an organic acid or an inorganic acid, and the organic acid includes, for example, formic acid, acetic acid, propionic acid, lactic acid, butyric acid, isobutyric acid, trifluoroacetic acid, malic acid, maleic acid, malonic acid, fumaric acid, succinic acid, succinic monoamide, glutamic acid, tartaric acid, oxalic acid, citric acid, glycolic acid, glucuronic acid, ascorbic acid, benzoic acid, phthalic acid, salicylic acid, anthranilic acid, dichloroacetic acid, aminooxyacetic acid, benzenesulfonic acid, p-toluenesulfonic acid, or methanesulfonic acid-based salts. The inorganic acid includes, for example, hydrochloric acid, bromic acid, sulfuric acid, phosphoric acid, nitric acid, carbonic acid or boric acid-based salts. The inorganic acid may be in the form of a hydrochloride or an acetate.

The aforementioned acid addition salts are prepared using a general salt preparation method performed by a) mixing the compound of Formula 1 or 2 directly with an acid; b) dissolving one of them in a solvent or a water-containing solvent and mixing the solution with the other one; or c) placing the compound of Formula 1 or 2 in a solvent or an acid in a water-containing solvent and mixing them.

Separately, in addition to the above, examples of possible salt forms include, but are not limited to, GABA salts, gabapentin salts, pregabalin salts, nicotinates, adipates, hemimalonates, cysteine salts, accetylcysteine salts, methionine salts, arginine salts, lysine salts, ornithine salts, or aspartates.

The present invention may provide the riboflavin of Formula 1 or the riboflavin analog of Formula 2 as a pharmaceutical composition for inhibiting or alleviating itching.

The term “itching” as used herein refers to a condition of the skin causing an unpleasant sensation that causes an urge to scratch the skin, and particularly, may refer to a histamine-induced symptom. Itching may be caused by stimulation such as light contact, temperature changes, stress, and the like, chemical, physical, and electrical stimulations, or the like, but the present invention is not limited thereto. Non-limiting examples of diseases accompanied by itching include skin diseases due to mites, lice, bug bites, urticaria, and the like, psoriasis, dry skin, senile itching, mycosis fungoides, atopic dermatitis, contact dermatitis, nummular eczema, neurodermatitis, and autoimmune disorders.

When the composition according to the present invention is used as a drug, the composition may further include one or more active ingredients exhibiting the same or similar functions. For example, the composition may include known ingredients for inhibiting or alleviating itching. When the composition includes additional active ingredients, the effect of the composition of the present invention may be further enhanced. When the above-described ingredients are added, skin safety, ease of formulation, and stability of active ingredients, according to the combined use, may be considered.

In addition, the pharmaceutical composition of the present invention may further include a pharmaceutically acceptable carrier.

The pharmaceutically acceptable carrier may include various ingredients such as a buffer, injectable sterile water, normal saline or phosphate-buffered saline, sucrose, histidine, a polysorbate, or the like.

The pharmaceutical composition of the present invention may be formulated into oral or parenteral preparations, and particularly, may be formulated into oral preparations, injections, mucosal preparations, inhalants, or percutaneous absorption preparations. For formulation, generally used diluents or excipients such as fillers, extenders, binders, wetting agents, disintegrating agents, surfactants, or the like may be used.

Examples of solid preparations for oral administration include tablets, pills, powder, granules, capsules, and the like, and these solid preparations may be formulated by mixing the pharmaceutical composition of the present invention with at least one excipient, for example, starch, calcium carbonate, sucrose, lactose, gelatin, or the like.

In addition to simple excipients, lubricants such as magnesium stearate and talc are also used. Examples of liquid preparations for oral administration include suspensions, liquids for internal use, emulsions, syrups, and the like, and these liquid preparations may include, in addition to simple commonly used diluents, such as water and liquid paraffin, various excipients, for example, a wetting agent, a sweetener, a flavoring agent, a preservative, and the like.

Preparations for parenteral administration include injections, mucosal preparations, inhalants, or percutaneous absorption preparations.

The pharmaceutical composition of the present invention may provide an effect of inhibiting or alleviating itching when an effective amount of the riboflavin of Formula 1 or the riboflavin analog of Formula 2 is included therein. As used herein, the term “inhibition” means all actions that reduce or delay itching via administration of the composition according to the present invention. In addition, the term “alleviation” as used herein means all actions that improve or beneficially change itching via administration of the composition according to the present invention.

The effective amount of the riboflavin of Formula 1 or the riboflavin analog of Formula 2 included in the composition of the present invention may vary depending on a formulated form of the composition, a method of application on the skin, residence time on the skin, and the like. For example, when the composition is commercialized as a drug product, the composition may include the compound at a higher concentration than in a case in which the composition is commercialized as a cosmetic which is generally applied on the skin. In one embodiment, the amount of riboflavin or an analog thereof when formulated into oral preparations may range from about 200 mg/kg to about 800 mg/kg or about 300 mg/kg to about 600 mg/kg, and the concentration of riboflavin or an analog thereof when formulated into intradermal injections may be 1.5 μM or less or 0 μM or less. In addition, the concentration of riboflavin or an analog thereof when formulated into percutaneous absorption preparations may be 10 mM or less or 0.5 mM or less.

The pharmaceutical composition of the present invention may be used alone or in combination with methods using surgery, radiation therapy, hormone therapy, chemotherapy, or biological response modifiers.

The present invention may also provide an external preparation for inhibiting or alleviating itching, which includes the riboflavin of Formula 1 or the riboflavin analog of Formula 2.

When the riboflavin or an analog thereof is used in an external preparation, the composition may further include an adjuvant commonly used in the dermatological field, such as any other component commonly used in external preparations for the skin, for example, a fatty material, an organic solvent, a dissolving agent, a concentrating agent, a gelling agent, a softening agent, an antioxidant, a suspending agent, a stabilizer, a foaming agent, a fragrance, a surfactant, water, an ionic or non-ionic emulsifying agent, a filler, metal ion sequestering and chelating agents, a preservative, a vitamin, a blocking agent, a wetting agent, essential oils, a dye, a pigment, a hydrophilic or lipophilic activating agent, a lipid vesicle. In addition, the above ingredients may be introduced in amounts generally used in the dermatological field.

When the composition is provided as an external preparation, the preparation may be, but is not limited to, ointment, patches, gels, creams, sprays, or the like.

The present invention may also provide a cosmetic composition for inhibiting or alleviating itching, which includes the riboflavin of Formula 1 or the riboflavin analog of Formula 2.

When the riboflavin or an analog thereof is used in cosmetics, the prepared cosmetics may be in the form of general emulsion or solubilized preparations. For example, the composition may be formulated into hypoallergenic cosmetics, skin care products, skin toner, nourishing lotions, nourishing creams, massage creams, essence, eye creams, cleansing creams, cleansing foams, cleansing water, pack, creams, essence, shampoo, hair conditioner, soaps, sprays, or powder.

In addition, the cosmetics may include an adjuvant commonly used in the cosmetic field, such as a fatty material, an organic solvent, a dissolving agent, a concentrating agent, a gelling agent, a softening agent, an antioxidant, a suspending agent, a stabilizer, a foaming agent, a fragrance, a surfactant, water, an ionic or non-ionic emulsifying agent, a filler, metal ion sequestering and chelating agents, a preservative, a vitamin, a blocking agent, a wetting agent, essential oil, a dye, a pigment, a hydrophilic or lipophilic activating agent, a lipid vesicle, or the like.

In one embodiment, the composition may include riboflavin or an analog thereof in an amount of about 0.0001 wt % to about 10 wt % (preferably about 0.0001 wt % to about 1 wt %) with respect to a total weight of the composition. When the composition of the present invention includes riboflavin or an analog thereof in an amount of less than 0.0001 wt %, it is difficult to expect an effect of sufficiently inhibiting or alleviating itching. When the amount of the riboflavin or an analog thereof is greater than 10 wt %, unwanted reactions such as allergies and the like may occur or skin safety problems may occur.

In addition, in the present invention, to enhance skin permeability of riboflavin or an analog thereof, the riboflavin or an analog thereof may be formulated into liposomes. Such a liposome preparation method may be carried out using a preparation method commonly used in the art.

Hereinafter, the present invention will be described in detail with reference to the following examples. These examples are provided for illustrative purposes only and are not intended to limit the scope of the present invention. These examples are provided so that the invention is complete and those of ordinary skill in the art to which the present invention pertains fully understand the scope of the present invention, and the present invention should be defined by the scope of the appended claims.

### EXAMPLES

### Preparation Example 1. Preparation of Drug Preparations of Riboflavin

Drug preparations including riboflavin as an active ingredient were prepared. In the case of preparations for oral administration, drug preparations including riboflavin at three concentrations (100 mg/kg, 300 mg/kg, or 600 mg/kg) were prepared, and in the case of local intradermal injections, riboflavin was added at a concentration of 1 μM/50 μl.

As a control, oral administration and local intradermal preparations were prepared so as to have the same composition as that used above, except that the riboflavin was not added.

### Experimental Example 1. Effect of Riboflavin on Histamine-Induced Itching (Preparations for Oral Administration)

(1) Method

An effect of inhibiting or alleviating itching was tested using the preparations for oral administration prepared according to Preparation Example 1. For the test, an animal behavioral test was performed using the mouse model disclosed in Non-Patent Document 6.

Groups each including 10 male C57BL/6 mice weighing 20 g to 23 g were used in the test. These groups were accommodated under control of a temperature of 23° C. to 25° C. The mice were freely fed food and water. For the itching-induced animal behavioral model test, the mice were orally administered the preparations for oral administration having different concentrations of riboflavin, which were prepared according to Preparation Example 1 (100 mg/kg, 300 mg/kg, or 600 mg/kg). In addition, the mice were treated with the control prepared according to Preparation Example 1.

To induce scratching, histamine, which is known to cause an itching sensation in humans, was used. At this time, histamine was dissolved in sterile physiological saline. 30 minutes after administration of the preparations for oral administration, 50 μl (500 μg/site) of histamine was injected intradermally into the back of the neck of each mouse. Subsequently, the movement of each mouse was observed for 30 minutes, and the number of scratches on the injected area with the hind leg was counted.

The results were analyzed by calculating the mean and standard deviation, and a statistical difference of mean values was determined using a Student's t-test.

(2) Results

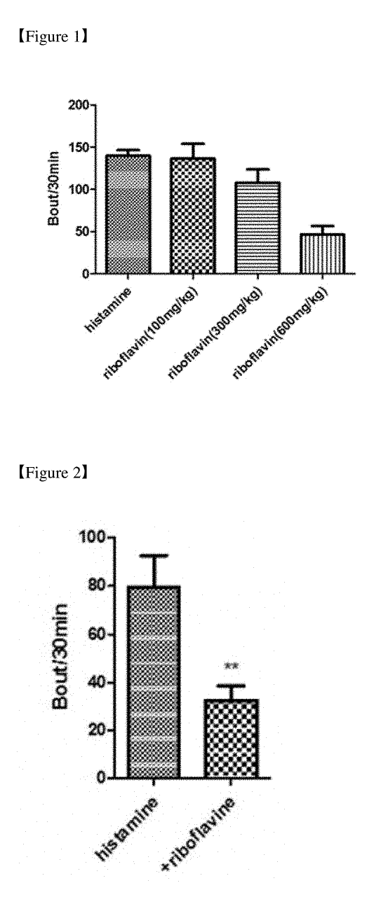

The results are illustrated as a graph in FIG. 1. FIG. 1 illustrates results of evaluating an effect of the preparations for oral administration including different concentrations of riboflavin on histamine-induced itching. In FIG. 1, histamine denotes a control.

As illustrated in FIG. 1, histamine-induced itching was caused in the control not including riboflavin, and itching responses were reduced in the cases where riboflavin was treated. In addition, an effect of riboflavin on inhibiting histamine-induced itching exhibited a dose-dependent pattern, and it was confirmed that the effect was further enhanced according to an increase in the concentration of riboflavin.

From the above results, it is determined that riboflavin effectively inhibits histamine-dependent itching and has a dose-dependent pattern.

### Experimental Example 2. Effect of Riboflavin on Histamine-Induced Itching (Local Intradermal Injection)

(1) Method

An effect of inhibiting or alleviating itching was tested using the local intradermal injection prepared according to Preparation Example 1.

The test was carried out in the same manner as in Experimental Example 1, except for the following. For the itching-induced animal behavioral model test, 1 μM/50 μl of the local intradermal injection prepared according to Preparation Example 1 was injected intradermally into the back of the neck of each mouse. In addition, upon administration of the preparation, 50 μl (100 μg/site) of histamine was injected intradermally at the same time.

(2) Results

The results are illustrated as a graph in FIG. 2. FIG. 2 illustrates evaluation results of histamine-induced itching upon co-administration of riboflavin and histamine. In FIG. 2, histamine indicates a control, and +riboflavin denotes a case in which the local intradermal injection was administered.

As illustrated in FIG. 2, while histamine-induced itching was unable to be significantly reduced in the control, itching responses were reduced in the case in which riboflavin was treated.

From the above results, it was confirmed that riboflavin was effective in inhibiting histamine-induced itching.

### Experimental Example 3. Effect of Riboflavin on Histamine-Induced Peripheral Nerve Activity

Histamine-induced itching is known to occur through peripheral nerve excitation. In the present experimental example, it was examined whether histamine-induced peripheral nerve activity was blocked by riboflavin.

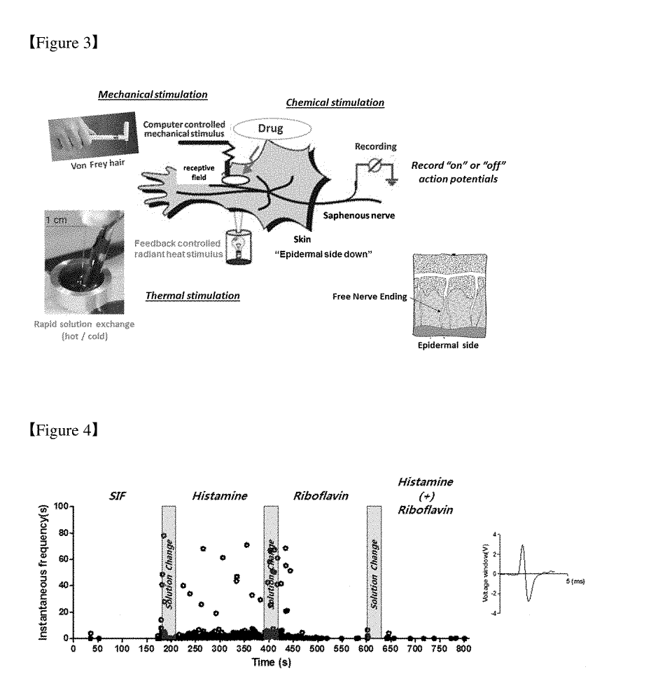

After securing preserved skin-afferent nerve tissue, histamine was administered to the sole of the foot to confirm nerve activity, and such histamine-induced nerve activity was blocked by riboflavin (see FIG. 3).

In addition, to confirm an effect of riboflavin on blocking histamine-induced peripheral nerve activity, it was examined using a membrane voltage clamp method whether a histamine-induced current was inhibited by riboflavin.

To this end, spinal cord dorsal root ganglion cells of each mouse were isolated and primarily cultured, and then electrophysiological characteristics of the spinal cord dorsal root ganglion cells were recorded using the membrane voltage clamp method.

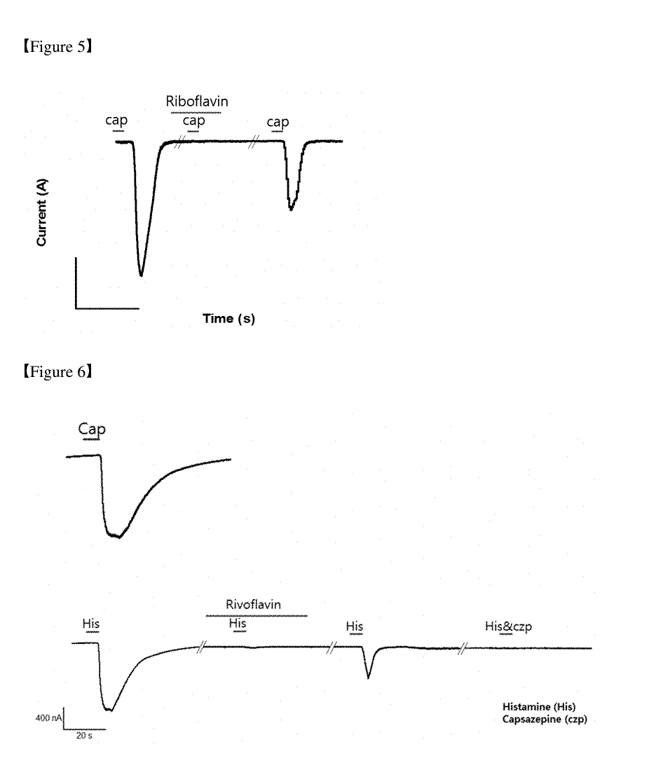

The results are illustrated as a graph in FIG. 4. FIG. 4 illustrates results of confirming that a current induced by histamine was inhibited by riboflavin using a membrane voltage clamp method. As illustrated in FIG. 4, it can be confirmed that histamine generates an inward current, and the current is blocked by riboflavin. This suggests that histamine generates an inward current in peripheral nerve cells and the current is specifically blocked by riboflavin.

### Experimental Example 4. Capsaicin Current Blocking Effect of Riboflavin in TRPV1-Expressing HEK293 Cells

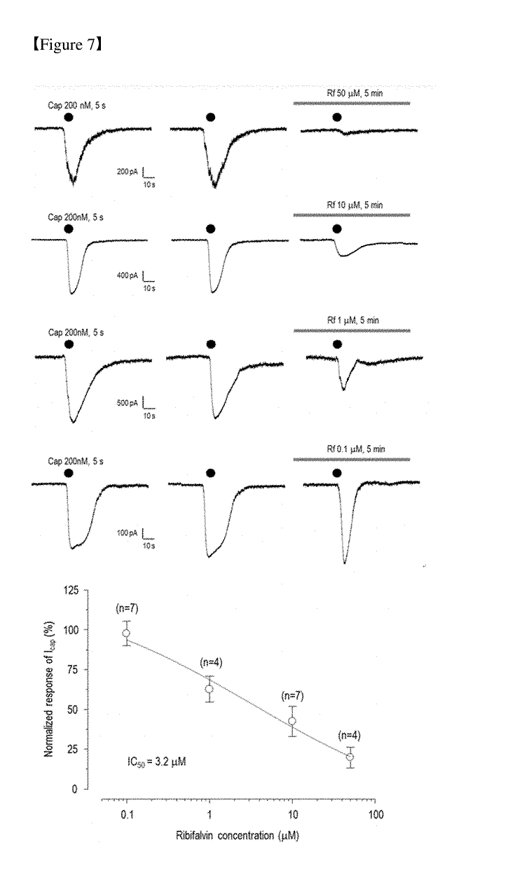

TRPV1 was expressed in HEK 293 cells to carry out a capsaicin current blocking experiment. Under these conditions, riboflavin excluded other channels and a direct effect of riboflavin was examined. The results are illustrated in FIG. 5. FIG. 5 illustrates results of confirming that a capsaicin-induced current was inhibited by riboflavin. As illustrated in FIG. 5, riboflavin exhibited a statistically significant effect of blocking channels in a concentration-dependent manner. However, 100 nM of riboflavin did not effectively block TRPV1 in the HEK 293 cells.

A degree to which the capsaicin-induced current was inhibited according to the concentration of riboflavin was expressed as a concentration-response curve, and as a result of calculating IC₅₀ by applying sigmoidal fitting, the IC₅₀ was 3.2 M. This means that riboflavin directly blocks TRPV1 in a concentration-dependent manner (see FIGS. 6 and 7).

### Preparation Example 2. Preparation of Drug Preparation of Roseoflavin

A preparation for transdermal administration including roseoflavin as an active ingredient was prepared.

In particular, 10% roseoflavin was mixed with Vaseline cream to prepare a preparation for transdermal administration. In the preparation, a final concentration of roseoflavin was 1 mM (40 μg/100 μl).

### Experimental Example 5. Effect of Roseoflavin on Histamine-Induced Itching

(1) Method

An effect of inhibiting or alleviating itching was tested using the preparation for transdermal administration prepared according to Preparation Example 2. For the test, an animal behavioral experiment was carried out in the same manner as in Experimental Example 1.

In particular, 30 minutes before 100 μg/50 μl of histamine was injected, the preparation for transdermal administration was applied on the skin, and then the preparation was removed 5 minutes before histamine was injected. Subsequently, histamine-induced itching was observed for 30 minutes.

(2) Results

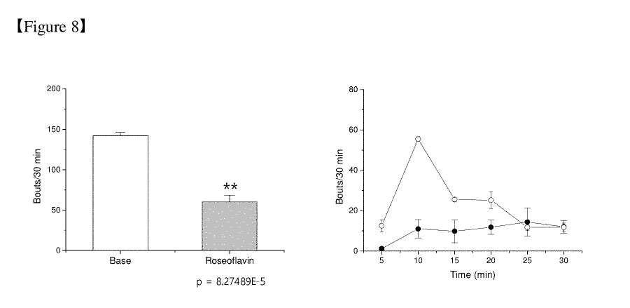

The results are illustrated as a graph in FIG. 8. FIG. 8 illustrates evaluation results of histamine-induced itching by roseoflavin. In FIG. 8, histamine denotes a control (roseoflavin-free Vaseline was used). The graph on the left side of FIG. 8 shows mean values for effects of blocking histamine-induced itching and itching when roseoflavin is treated, and the graph on the right side of FIG. 8 shows changes in itching over time.

As illustrated in FIG. 8, histamine-induced itching was caused in the control not using roseoflavin, and itching responses were reduced in the case in which roseoflavin was treated, and it can be particularly confirmed that itching behavioral responses were reduced to 60% (142 times down to 60 times). This suggests that riboflavin or an analog thereof, i.e., roseoflavin, which is percutaneously applied, will relieve histamine-induced itching.

The riboflavin according to the present invention is very effective in inhibiting or alleviating itching, scalp itching, or allergic itching by inhibiting itching information transmission through blocking of a current generated by histamine, which is a major factor of itching.

As is apparent from the foregoing description, a composition for inhibiting or alleviating itching, which includes riboflavin or an analog thereof inhibits itching information transmission through blocking of a current generated by histamine, which is a major factor of itching. Through such a pharmacological action mechanism, the riboflavin or an analog thereof is very effective in inhibiting or alleviating itching, scalp itching, allergic itching, or the like.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the invention as defined by the following claims.

What is claimed is:

1. A pharmaceutical composition for inhibiting or alleviating itching, the pharmaceutical composition comprising riboflavin represented by Formula 1 below or a riboflavin analog represented by Formula 2 below:

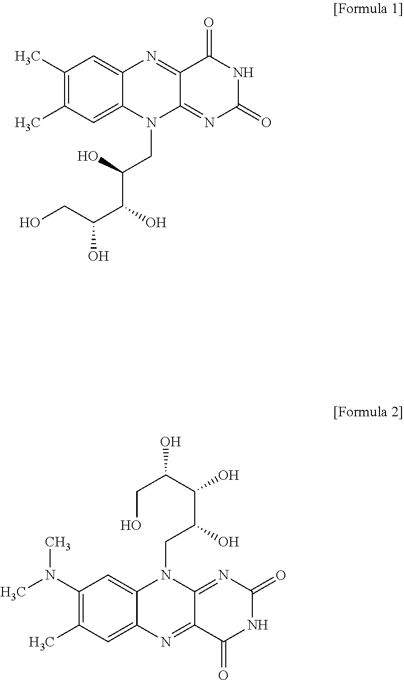 [SMILES: Cc1cc2nc3c(=O)[nH]c(=O)nc-3n(C[C@H](O)[C@H](O)[C@H](O)CO)c2cc1C ; Cc1cc2nc3c(=O)[nH]c(=O)nc-3n(C[C@@H](O)[C@@H](O)[C@@H](O)CO)c2cc1N(C)C]

2. The pharmaceutical composition of claim 1, wherein the pharmaceutical composition is formulated into an oral preparation, an injection, a mucosal preparation, an inhalant, or a percutaneous absorption preparation.

3. The pharmaceutical composition of claim 2, wherein, when formulated into the oral preparation, an amount of the riboflavin or the riboflavin analog is in a range of about 200 mg/kg to about 800 mg/kg.

4. The pharmaceutical composition of claim 2, wherein, when formulated into the injection, a concentration of the riboflavin or the riboflavin analog is 1.5 μM or less.

5. The pharmaceutical composition of claim 2, wherein, when formulated into the percutaneous absorption preparation, a concentration of the riboflavin or the riboflavin analog is 10 mM or less.

6. An external preparation for inhibiting or alleviating itching, the external preparation comprising riboflavin represented by Formula 1 below or a riboflavin analog represented by Formula 2 below:

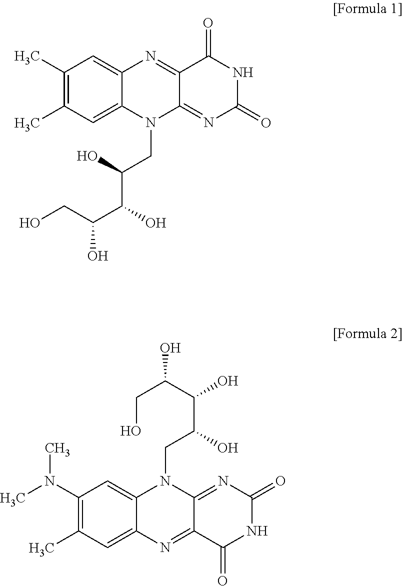 [SMILES: Cc1cc2nc3c(=O)[nH]c(=O)nc-3n(C[C@H](O)[C@H](O)[C@H](O)CO)c2cc1C ; Cc1cc2nc3c(=O)[nH]c(=O)nc-3n(C[C@@H](O)[C@@H](O)[C@@H](O)CO)c2cc1N(C)C]

7. The external preparation of claim 6, wherein the external preparation is formulated into an ointment, a patch, a gel, a cream, or a spray.

8. A cosmetic composition for inhibiting or alleviating itching, the cosmetic composition comprising riboflavin represented by Formula 1 below or a riboflavin analog represented by Formula 2 below:

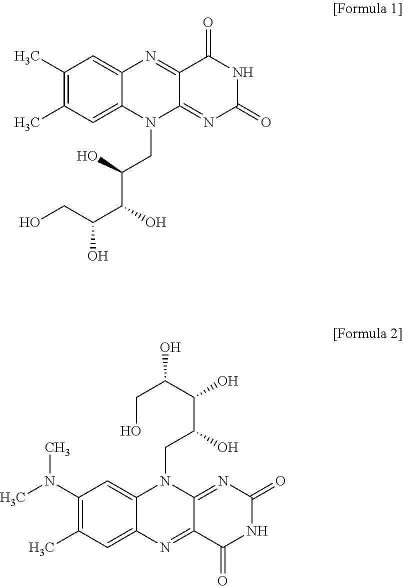 [SMILES: Cc1cc2nc3c(=O)[nH]c(=O)nc-3n(C[C@H](O)[C@H](O)[C@H](O)CO)c2cc1C ; Cc1cc2nc3c(=O)[nH]c(=O)nc-3n(C[C@@H](O)[C@@H](O)[C@@H](O)CO)c2cc1N(C)C]

9. The cosmetic composition of claim 8, wherein the cosmetic composition is formulated into a hypoallergenic cosmetic, a skin care product, a skin toner, a nourishing lotion, a nourishing cream, a massage cream, an essence, an eye cream, a cleansing cream, a cleansing foam, cleansing water, a pack, a cream, a shampoo, a hair conditioner, a soap, a spray, or powder.
